(19) United States
(12) Patent Application Publication
(10) Pub. No.: US 2026/0207938 A1
(43) Pub. Date: Jul. 23, 2026

(54) SYSTEM AND METHOD FOR ACTIVATION OF CUTANEOUS SENSORY FIBERS USING STIMULATION WAVEFORMS

(71) Applicant: Algiamed Technologies LTD, Burnaby (CA)

(72) Inventors: Lukas Linde, Vancouver (CA); Paul Potgieter, Coquitlam (CA); Corlius Birkill, Garibaldi Highlands (CA)

(73) Assignee: Algiamed Technologies LTD, Burnaby (CA)

(21) Appl. No.: 19/451,666

(22) Filed: Jan. 16, 2026

Related U.S. Application Data
(60) Provisional application No. 63/746,617, filed on Jan. 17, 2025.

(51) Int. Cl. A61N 1/36; A61N 1/04

(52) U.S. Cl. CPC A61N 1/36034; A61N 1/0456; A61N 1/0476; A61N 1/0492; A61N 1/36021

(57) ABSTRACT

A system and method for applying electromagnetic signals to activate desired nerve fibers.

### CROSS-REFERENCE TO RELATED APPLICATIONS

The present application claims benefit of and priority to U.S. Provisional Patent Application Ser. No. 63/746,617 filed Jan. 17, 2025 entitled SYSTEM AND METHOD FOR ACTIVATION OF CUTANEOUS SENSORY FIBERS USING STIMULATION WAVEFORMS, the entire content of which is incorporated by reference herein.

### FIELD OF THE DISCLOSURE

The present disclosure relates to activation of cutaneous sensory fibers (i.e. Aβ, Aδ and C fibers) using electromagnetic waveforms. In particular, the present disclosure relates to multiple electrode arrays that provide transcutaneous electrical stimulation to sensory nerve fibers.

### RELATED ART

Transcutaneous electrodes and associated electrical stimulation devices are known to be used to provide different treatments in the human body. For example, transcutaneous electrical nerve stimulation (TENS) is commonly used for the treatment of pain and relies on principles of gate control theory. Briefly, peripheral sensory neurons located within the skin, which transmit information about the sense of touch are mediated by large, myelinated Aβ sensory afferents. Meanwhile, noxious stimuli related to actual or potential tissue damage, are mediated by nociceptive afferents also located in the skin and characterized by both thinly myelinated Aδ afferents, and small unmyelinated C fibers. The notion of gate control relies on the principle that activation of larger, faster (in terms of signal transduction) Aβ sensory fibers reaches the spinal cord and subsequent higher order processing centers of the nervous system before slower nociceptive (Aδ and C) fibers. This faster transduction of Aβ sensory fibers effectively “closes the gate”, and blocks transmission of nociceptive signals that give rise to pain sensation. This premise forms the

basis of the use of TENS to manage pain, as is well known. While TENS is typically effective for pain management, the use of TENS electrodes for the transcutaneous electrical stimulation of peripheral nerve fibers is significantly limited in terms of the method of activation. Namely, activation thresholds of peripheral sensory nerve fibers vary in line with nerve afferent size, such that tactile afferents (Aβ) are activated with lower stimulation intensity compared to smaller nociceptive (Aδ and C) afferents, when using standard transcutaneous electrodes. For the purposes of TENS, this is effective, as pain relief is based on the aforementioned gate control theory. However, if the purpose of transcutaneous electrical stimulation is to specifically activate smaller diameter nociceptive (Aδ and C) afferents, standard transcutaneous electrical stimulation is severely limited. Only by progressively increasing stimulation intensity will activation of nociceptive afferents begin, however this will remain concomitant with activation of tactile Aβ sensory fibers as well. Thus, for the purposes of selective activation of nociceptive afferents only, for either diagnostic or therapeutic benefit, alternative approaches to transcutaneous electrical stimulation are desired.

A critical aspect of selective activation of tactile and nociceptive cutaneous sensory receptor subtypes is their location within the skin, and activation kinetics. The skin may be divided into three broad layers, the most superficial being the epidermis, under which is the dermis, followed by subcutaneous layers. Cutaneous Aδ and C fibers, which transmit nociceptive information, terminate as free nerve endings in the mid-layer of the epidermis (the most superficial layer). Conversely, Aβ cutaneous fibers terminate into either hair follicles or specialized sensory organelles (especially in glabrous skin of the palms of the hands and soles of the feet), all located at varying depths within the dermis of the skin. Despite being deeper within the skin, it takes notably less input voltage/energy from traditional transcutaneous electrical stimulation to activate Aβ fibers compared to Aδ and C fibers. This is due primarily to the larger size and lower rheobase of Aβ fibers compared to the smaller Aδ and C fibers. Practically speaking, while Aδ and C fibers are more superficial, and thus closer to any stimulation device applied transcutaneously, the lower rheobase and larger size of Aβ fibers explains their preferential responsiveness to such stimulation approaches. To overcome this limitation of traditional transcutaneous electrical stimulation, numerous efforts have been made to design electrodes that may better activate Aδ and C fibers in the most superficial layers of the skin, such as concentric ring electrodes, and percutaneous electrode arrays. Percutaneous electrodes, for example, which penetrate the most superficial layers of the skin can be oriented to allow shallow electrical fields, thereby preferentially activating those most superficial cutaneous fibers (Aδ and C). However, percutaneous designs are then limited in their ability to selectively activate Aβ fibers, which require reverting to simple transcutaneous electrode configurations.

In addition to differences in size, depth, and activation kinetics of Aβ, Aδ, and C fibers, various specialized sensory fiber subtypes exist within each of these three anatomical classes, as they pertain to tactile and nociceptive somatosensation. First, Aβ fibers are subdivided into various specialized sensory afferents, and specialized end organs, based on their location within glabrous or hairy skin. Generally, glabrous skin is provided on the palms of the hand, soles of the feet, lips, and regions of the genitalia. Aβ fibers are responsive to light touch and include low threshold mechano-receptive (LTMR) sensory afferents, with slowly adapting (SA) and rapidly adapting (RA) fibers. each including two specialized sensory end organs per fiber type (i.e. RAI, RAII, SAI, SAII). RAI fibers terminate into Meissner corpuscles, located at the top of the dermal papillae, forming close relationships with resident lamellar cells in the epidermis (top layer of the skin). Meissner corpuscles respond rapidly to skin indentation and are sensitive to vibration. RAII fibers terminate into Pacinian corpuscles, found deeper in the dermal layer of the skin. Pacinian corpuscles are sensitive only to the dynamic phases of indentation and are uniquely sensitive to high-frequency vibrations. SAI fibers terminate into Merkel cell complexes, and have frequency dependence in their force sensitivity, providing input on curvature of objects due to pressure sensitivity of various Merkel cell structures terminating in single SAI afferent fibers. SAII fibers terminate into Ruffini endings, primarily responsible for skin stretch sensations. All-together, glabrous skin contains very specific sensory organelles that encode mechanical stimulation into electrical activity of slowly adapting and fast adapting Aβ sensory neurons. In hairy skin, touch sensations are facilitated primarily by LTMR found surrounding hair follicles. Along with Aβ LTMR fibers, Aδ-LTMR fibers and C-LTMR fibers are also found surrounding hair follicles. Further specialization of Aβ LTMR fibers includes Aβ SAI-LTMR, Aβ RA-LTMR, and Aβ field-LTMR receptors. Aβ RA-LTMR are functionally similar to glabrous Meissner corpuscles in their signal transduction of rapid adapting on/off responses. Aβ SAI-LTMR form “touch domes” surrounding hair follicles, similar in function to Merkel cells in glabrous skin. Aβ field-LTMR, while highly sensitive to gentle stroking across the skin, are relatively insensitive to skin indentation and thus exhibit indentation responses characteristic of high-threshold mechanoreceptors. The rates of adaptation vary among the LTMRs, with the Aβ RA-LTMRs and Aδ-LTMRs adapting the fastest and the Aβ SAI-LTMRs the slowest. The Aβ field-LTMRs adapt rapidly at the lowest indentation forces for which they do respond, while at high forces, the responses of the Aβ field-LTMRs to indentation steps are more intermediately adapting and lack an off response. Aδ-LTMR and C-LTMR afferents surround hair follicles, and contribute to crude sense of touch. To this end, differentiation of Aβ, Aδ, and C sensory neurons within hairy skin is not solely dependent on the sensations detected (i.e. touch vs nociception and pain), but the timing of signal transduction (based on size of sensory receptor) is a key factor in verifying the successful stimulation of peripheral sensory neurons.

Nociceptors, the sensory neurons that detect potentially harmful stimuli, may also be categorized into various subtypes. First, nociceptive afferents are made up of both Aδ and C fiber sensory afferents such that there is variation in the relative speed that nociceptive stimuli are transmitted from the periphery to central pain processing centers. Aδ fibers are thinly myelinated and transmit signals quickly (relative to unmyelinated C fibers), contributing to the initial sharp sensation of pain. Conversely, C fibers are unmyelinated and transmit signals more slowly, leading to the lingering, dull, or throbbing pain that follows the initial sharp pain. As a general rule, A-fiber nociceptors do what C-fiber nociceptors do, but do it more robustly. They respond at higher discharge frequencies, and provide more discriminable information to the CNS. Encompassing both C and Aδ fibers, there are broad classes of nociceptive afferents based on the types of stimuli which they respond to. These classes include polymodal nociceptors that respond to mechanical, thermal, and chemical stimuli; mechano-thermal nociceptors that respond to both mechanical and thermal stimuli; thermoreceptive nociceptors that respond to extreme temperatures; and mechanically-insensitive or silent nociceptors that do not respond to mechanical stimulation (except under very high mechanical forces) but may respond in the presence of inflammation. In most early studies of nociceptors, only heat and mechanical stimuli were used to study nociceptors. Therefore, the nomenclature of CMH and AMH is often used to refer to C-fiber mechano-heat-sensitive nociceptors and A-fiber mechano-heat-sensitive nociceptors, respectively. Furthermore, if a fiber responds to heat and mechanical stimuli, the fiber will, in most cases, respond to chemical stimuli as well. Thus, CMHs and AMHs are often referred to as polymodal nociceptors, making the terms relatively interchangeable. In addition, nociceptors can be further characterized into two broad categories: peptidergic and non-peptidergic, based on the types of neurotransmitters they release. Peptidergic nociceptors release neurotransmitters like substance P and CGRP, known to contribute to states of peripheral and central sensitization. Finally, recent studies have sought to further classify nociceptor subtypes based on their molecular and functional taxonomy-which relies in part on the types of receptors present within specific nociceptors (along with their function). Here, a recent review outlined classes of nociceptors and pruriceptors based on the taxonomy of mouse dorsal root ganglions. Their findings align well with nociceptor sub-classifications previously discussed, insofar as they describe six classifications of C-fiber (C-cold, C-mechano-heat (likely polymodal), C-LTMR, and 3 of which appear to be both pruriceptive/warmth specific), and two main classifications of Aδ fiber related to nociception (being high threshold heat mechanoreceptors and heat receptors (i.e. mechanical insensitive)) (Kupari J, Ernfors P. Molecular taxonomy of nociceptors and pruriceptors. PAIN. 2023; 164 (6):1245. doi:10.1097/j. pain.0000000000002831). These taxonomy-based classifications overlap nicely with aforementioned classifications of C-fibers (CMH-polymodal, C-MIA, C-thermosensitive) and Aδ fibers (i.e. Type I mechanosensitive and Type II-mechanosensitive).

Cutaneous nociceptive afferents are found within the epidermis, approximately midway within the layers of keratinocytes. Typically, nociceptive free nerve endings exit the dermis and branch 2-5 times upon entering the epidermis. For Aδ fibers, the afferents shed their myelin sheath upon entering the epidermis, as such, Aδ and C fibers in the epidermis both appear as unmyelinated free nerve endings. Interestingly, while the conduction velocity of small myelinated Aδ fibers are, by definition, faster than that of unmyelinated C fibers, the terminal cutaneous branches of nociceptive Aδ fibers may conduct at a velocity characteristic of unmyelinated fibers due to demyelination during epidermal branching. These free nerve endings contain numerous channels, dependent upon the classification/responsiveness of each nociceptive afferent, that respond to different stimuli in the periphery. For example, TRPV1 channels-found on mechano-heat sensitive nociceptors, respond to noxious heat above 41 C, along with capsaicin. The location of nociceptive free nerve endings in the epidermis is substantially closer to the skin surface than tactile Aβ receptors, which are located at varying depths within the dermis.

Anatomical factors of cutaneous sensory afferents notwithstanding, further care is required in selective activation with respect to the frequency characteristics and stimulation polarity to target specific cutaneous sensory afferent subtypes. Prior studies have demonstrated partial efficacy in selectively stimulating one or more cutaneous sensory afferent fiber subtypes, however, no prior studies have demonstrated the ability to selectively activate all cutaneous sensory afferent subtypes, using transcutaneous multi-electrode arrays, solely by modifying active electrode sizes, shapes, field densities, polarities, carrier frequencies and stimulation rates. Further, unique properties of cutaneous nociceptive afferents may be leveraged to verify their selective activation via stimulation at specific frequencies, through sensory mapping (i.e. hot vs cold sensations in response to differing stimuli).

Nociceptive afferents, Aδ and C fibers, display both spatial and temporal summation of stimuli from the periphery. Temporal summation, often termed wind-up in animal models, describes the unique property of nociceptive afferents whereby repeated stimulation at specific frequencies (often ~1 Hz) results in an amplification of action potentials propagated towards the spinal cord. This unique and reversible property of nociceptive afferents is used to verify their activation (i.e. LTMR does not display temporal summation). Spatial summation describes the regional integration of nociceptive stimuli at the level of the spinal cord from varying sources. Here, increased nociceptive activity from multiple, neighboring sites can lead to an amplification of excitability in the dorsal horn of the spinal cord, due to convergence of sensory afferents.

Verification of the selective activation of Aβ, Aδ, and C fibers relies either upon verbal feedback of sensation from human participants, acquisition, analysis, and interpretation of some biological signal, or a combination of both. In the prior art, feedback on qualitative aspects of sensation following electrical stimulation may be used to verify cutaneous sensory fibers involved. For example, a light tapping sensation would infer Aβ involvement, while burning sensations would infer Aδ, and C fibers. Pragmatically, such feedback mechanisms are not suitable for use in a closed loop system

Accordingly, there is a need to provide a system that allows for activation and monitoring of activation of cutaneous sensory fibers, including Aβ, Aδ, and C fibers individually that addresses the problems discussed above.

### SUMMARY

In embodiments, it is an object of the present disclosure to provide a multichannel electrode array that is able to selectively control the size, shape, and depth of the electrical field to position it to selectively activate Aβ, Aδ, and C fibers.

Further, it is an object of the present disclosure to provide a system to selectively activate cutaneous sensory receptors, tactile or nociceptive, via electrical stimulation, while monitoring biological signals that are modulated in response to stimulation of said sensory receptors. Numerous transcutaneous and percutaneous multichannel stimulation electrodes of various sizes and configurations are used to selectively activate cutaneous sensory neurons. In embodiments, an electronic stimulation device provides and includes the ability to modulate waveforms of various stimulation electrodes within multi-channel stimulation arrays in order to target specific cutaneous sensory receptors.

In embodiments, multiple biosignals may be monitored and recorded, for example, electromyography(EMG)/acceleromyography(AMG) or electroencephalography (EEG) which may be acquired and analyzed in real time and may be utilized in semi-closed and fully closed feedback loop functionality of the stimulation system for generating and applying an electrical signal to activate nerve fibers.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 is a schematic representation of a multiple channel electrode array in accordance with an embodiment of the present disclosure;

FIG. 2 illustrates an exemplary illustration of a ring electrode for use in the array of FIG. 1 in accordance with an embodiment of the present disclosure;

FIG. 3 illustrates the ring electrode of FIG. 2 indicating a focal point of the ring electrode in accordance with an embodiment of the present disclosure;

FIG. 4 illustrates a plurality of ring electrodes that may be used in the array in accordance with an embodiment of the present disclosure; and

FIG. 5 illustrates an exemplary stimulator device configured to provide a desired waveform to the array of electrodes in accordance with an embodiment of the present disclosure.

FIGS. 6A-6C illustrate an exemplary electrode array suitable for use on a patient's leg in accordance with an embodiment of the present disclosure;

FIG. 6D illustrates the exemplary electrode array applied to a patient's leg;

FIGS. 7A-7C illustrate an exemplary electrode array suitable for use on a patient's arm in accordance with an embodiment of the present disclosure;

FIG. 7D illustrates the exemplary electrode array applied to a patient's arm;

FIGS. 8A-8C illustrate an exemplary electrode array suitable for use on a patient's foot in accordance with an embodiment of the present disclosure; and

FIG. 8D illustrates the exemplary electrode array applied to a patient's foot.

### DETAILED DESCRIPTION OF THE EXEMPLARY EMBODIMENTS

There have been numerous attempts to provide for stimulation of nerve fibers however, conventional attempts fail to provide for the ability to selectively target different nerve fibers. The system and method of the present disclosure allows for selective stimulation of different types of nerve fibers. In embodiments, this selective stimulation may be used to diagnose and/or treat neurologic conditions. In embodiments, the selective stimulation may be used to provide tactile feedback, for example, in a virtual reality experience or system. In embodiments, the selective stimulation of different nerve fibers may be used to simulate virtually any feeling or sensation.

In embodiments, the system 10 of the present disclosure provides for closed loop or partially closed loop feedback. In embodiments, such a system would benefit from real time monitoring of stimulation. In embodiment, three key biological signals may be monitored to provide active monitoring of physiological responses to electrical stimuli and are used to verify the activation of cutaneous sensory neuron subtypes. Electromyography (EMG) may be used to quantify electrical activity related to muscle contraction, and when paired with cutaneous electrical stimulation, may be used to assess cutaneous and nociceptive based reflexes, respectively. Briefly, based on aforementioned differences in size and speed of Aβ and Aδ cutaneous neurons, electrical stimulation triggers RII (tactile) and RIII (nociceptive) reflexes at differing latencies, respectively. In embodiments, by recording from selective muscles using EMG, it is possible to ascertain whether transcutaneous electrical stimulation evokes tactile, nociceptive, or some combination of reflexive response. Such responses, however, are limited to local spinal reflex pathways, and are not feasibly acquired from all possible locations of electrical stimulation.

In embodiments, electroencephalography (EEG) may provide a more global sensory system feedback system to cutaneous electrical stimulation in the peripheral nervous system. Briefly, EEG involves recording electrical activity from brain regions through the use of electrodes positioned along the scalp. Depending on the location, and number of recording EEG electrodes, activity related to sensation and pain perception may be acquired. Evoked potentials describe averaged EEG responses to repeated stimuli, and have long been employed for both tactile and nociceptive stimuli respectively. Similar to EMG based reflexes, latency differences in tactile vs nociceptive evoked potentials are also present and may also be used to differentiate between tactile and nociceptive peripheral sensation respectively. Further, resting state EEG and more elaborate EEG processing techniques, such as gamma band oscillations, may be associated with pain responsiveness. Thus, in embodiments, EEG based biosignals may provide sufficient biofeedback in a closed loop system to ensure selective stimulation of Aβ, Aδ, and C fibers, respectively.

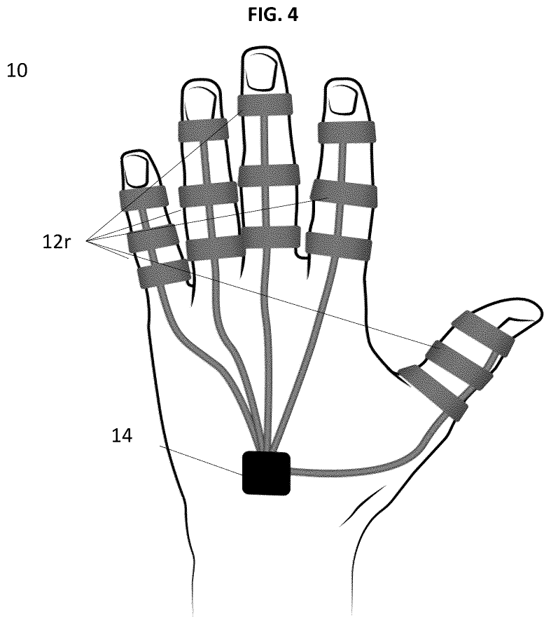

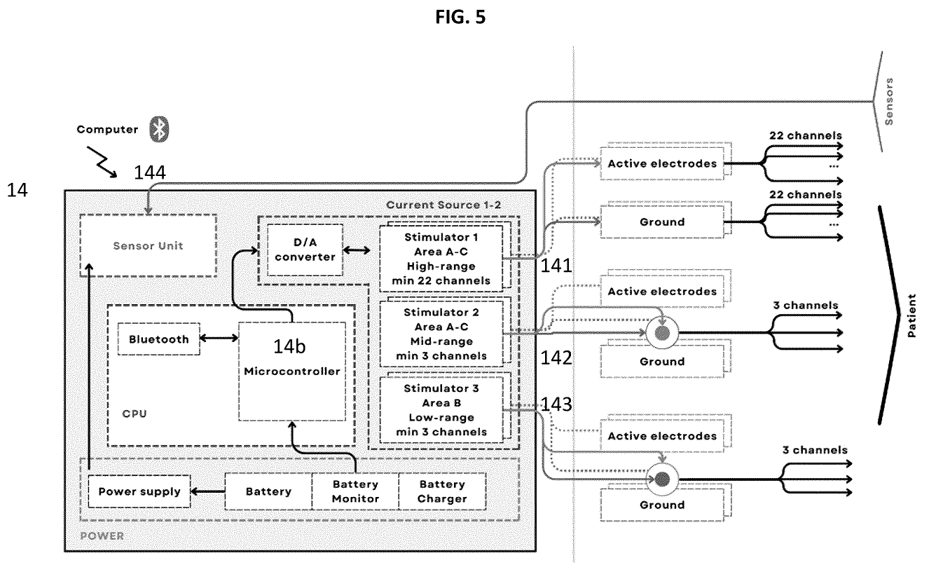

In embodiments, the system 10 (FIG. 4, for example) of the present disclosure uses one or more transcutaneous, multichannel electrode arrays 12, paired with an electrical stimulation device 14 that delivers customized waveforms, to selectively activate Aβ, Aδ, and C fibers, respectively. In embodiments, the system 10 may use a wide array of complex and purpose derived waveforms to selectively activate, or inactivate (i.e. block) cutaneous sensory afferent fiber subtypes, including Aβ, Aδ, and C fibers. In embodiments, multiple biological signal acquisition systems represented by the “sensors” illustrated in FIG. 5, for example, may be used to assess and monitor responses to the electrical stimulation and provided feedback to the stimulation device 14 to modulate electrical stimulation in both closed and semi-closed loop systems.

In embodiments, the system 10 of the present disclosure a) selectively activates Aβ, Aδ, and C fibers and b) incorporates novel electrical stimulation paradigms for selective peripheral fiber activation, c) incorporates therapeutic radiofrequency stimulation within stimulation paradigms, d) enables objective assessment of radiofrequency stimulation efficacy based on biosignal acquisition and e) incorporates a closed-loop feedback system

to verify the selective stimulation of cutaneous Aβ, Aδ, and C fibers via EMG/AMG and/or EEG biosignal monitoring.

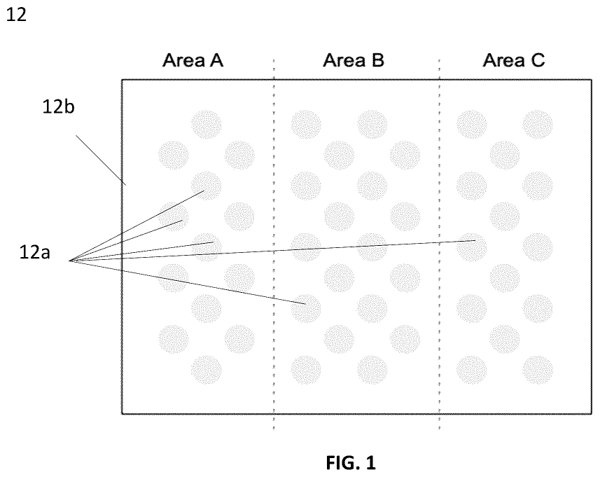

In embodiments, the system 10 of the present disclosure includes transcutaneous and percutaneous multichannel stimulation electrodes 12a provided in an array 12 (see FIG. 1, for example) that may be hemispherical in shape. In embodiments, the electrodes 12a may also be square, circular, rectangular or any other shape. In embodiments, the array 12 may be 1 cm by 1 cm and may be up to 10 cm by 10 cm in size. In embodiments, the array 12 may be larger, if desired. In embodiments, the number of individually controlled electrodes 12a within array 12 may vary, with up to 50 electrodes in the largest configuration, and 3 in the smallest. In embodiments, the number of individually controlled electrodes may be more than 50, if desired. In embodiments, the electrodes 12a may be spaced apart by 0.5 mm to 1.0 cm. In embodiments, variations in electrode size and shape and individual electrode density may be varied depending on application of the electrode array 12 to a particular part of the body. In embodiments, variations in electrode size, shape, and individual electrode density may be used to overcome regional variations in skin thickness and innervation density of specific cutaneous tactile and nociceptive sensory neurons of interest.

In embodiments, stimulation waveforms may be delivered to the multichannel electrodes 12a from electronic stimulation device 14 (See FIGS. 4-5, for example). In embodiments, the electronic stimulation device 14 may deliver stimulation waveforms to one or more ring electrodes 12r as indicated in FIG. 4, or any other electrode. In embodiments, the waveforms may include half sine waves, sine waves, square waves, pulses or other waves (e.g., ramps) with varying rise and fall times. In embodiments, pulse widths may be as short as 0.1 μs and may be as long as 500 ms. In embodiments, the frequency of stimulation ranges from 0.1 Hz up to 1 MHz. In embodiments, carrier frequencies of up to 50 MHz may be used in concert with nominal frequencies. In embodiments, amplitudes range from 0.01 mA up to 30 mA. In embodiments, the polarity of stimuli may be cathodic or anodic, depending on the number and regional variation of active electrodes 12a within the array, or may vary according to some input function.

In embodiments, the electrode array 12 may include a flat, rectangular electrode patch 12b and/or one to five ring electrodes 12r (see FIG. 4). In embodiments, the patch 12b and ring electrodes 12r may be used together or separately, depending on the application. In embodiments, rectangular patch 12b may include three to forty electrodes 12 a of diameter 0.1 mm to 1 mm each. In embodiments, additional electrodes may be used. In embodiments the electrodes 12a on patch 12b are divided into three sections lengthwise. In embodiments, more than one electrode array patch, as described in FIG. 4, may be used to stimulate multiple body regions simultaneously or in close temporal proximity. For example, both hands or both feet may be stimulated simultaneously or in close temporal proximity to explore side-to-side differences in sensation. Further, multiple electrode arrays may be placed on opposing sides of limbs, for example, to enable for nerve stimulation across larger regions. In embodiments, the electrode array 12 may be provided on a cloth. In embodiments, the patch 12b may be provided on the cloth. In embodiments, the electrodes 12a may be provided directly on the cloth such that the cloth acts as the pad 12b. In embodiments, the electrode array 12 may be provided on a garment that is worn by the patient. In one non-limiting example, the electrode array 12 may be provided on a sock worn by a diabetic patient. A common occurrence in diabetics is neuropathy in the foot. In embodiments, the electrode array 12 may be provided in the patient's sock and may be used both for diagnostic and therapeutic purposes. That is, selective activation of nerve fibers using the array 12 may be used to diagnose neuropathy in the user's foot and/or to treat it.

In embodiments, Area A (FIG. 1) may include zero to two thirds of the total number of electrodes 12a and may include hemispherical, flat or circular electrodes. In embodiments, Area B includes zero to one half of the total number of electrodes 12a. In embodiments, the electrodes 12a in Area B include sharp, conical tips that may pierce the stratum corneum and may be surrounded by non-penetrating electrodes. In embodiments, Area C may include zero to one half of the total electrodes which may be hemispherical or flat circular electrodes.

In embodiments, the width of the electrode array 12 is between 1 cm and 3 cm, and the length is between 2 cm and 10 cm. In embodiments, in each section, each row of the electrodes is alternately spaced, as indicated in FIG. 1, for example.

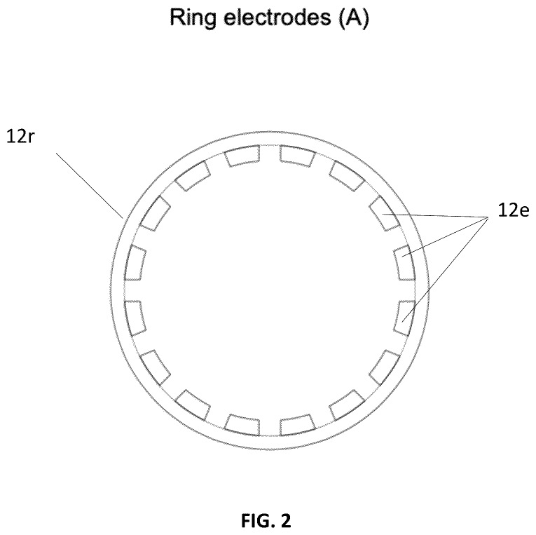

In embodiments, the ring electrodes 12r (see FIG. 2, for example) may include three to twenty electrode elements 12e placed on the inside of a ring of a malleable material that may conform to the shape of the finger as generally illustrated in FIG. 2.

In embodiments, all of the electrode elements 12a, 12r are attached to electrical stimulation device 14. In embodiments, the electrical stimulation device 14 may provide sinusoidal signals, half sinusoidal (in other words, the absolute value of a sinusoidal signal) signals, sinusoidal signals with a half-period pause (i.e., the positive part of a sinusoidal function), square/rectangular waves of equal or differing positive and negative amplitudes, or pulses, or ramped waveforms. In embodiments, regardless of wave shape, amplitude may vary from 0.01 mA to 30 mA. In embodiments, frequency may vary from 0.1 Hz to 1 MHz.

### Areas A and C

Areas A and C are functionally relevant for the selective activation of large touch fiber subtypes (Aβ), of small, myelinated fibers (Aδ), and pre-conditioning a fiber block. In embodiments, the protruding electrodes in Area B enhance the skin-electrode interface to deliver a high current density in the proximity of nociceptive endings—functionally relevant for the selective activation of small unmyelinated nerve fibers (C fibers).

The multi-channel electrode array 12 may selectively activate small myelinated fibers (Aδ). In embodiments, a central electrode within a group of electrodes acts as the cathode and surrounding electrodes provide anodic stimulation. By varying the current distribution in the surrounding electrodes, nerves that are not situated directly underneath the central cathode may be activated. Several clusters of electrodes may be utilized to increase the number of nerves being activated, or to ensure effective activation of a single nerve fiber.

In embodiments, the multi-channel electrode array 12 may selectively block large myelinated fibers (Aβ). Surface electrodes in Areas A and C may induce a pre-conditioning fiber block by using specific stimulation settings (see STIMULATION SETTINGS) and electrode configurations. In embodiments, the activation of Areas A and C, as represented in FIG. 1, is one of the recommended configurations for fiber blocking. In embodiments, blocking may be provided by positioning a ground electrode opposite from the stimulated area.

### Area B

In embodiments, the multi-channel electrode array 12 can selectively activate small unmyelinated fibers (C-fibers). Because of the orientation of the fibers in the skin, one percutaneous electrode is designated as anodic, and the surrounding (flat or hemispherical) electrodes are cathodic, or vice versa. Area B may also be used to initiate the blocking of certain fibers while areas A and/or C are active in stimulating other fibers.

### Ring Electrodes

In embodiments, ring electrodes 12r allow for advanced electric field shaping due to their circular geometry. Specifically, in embodiments, in order to activate Aβ fibers at depth without activating Aδ or C fibers, electrodes 12r may be positioned on one side of a given fiber and may be used to constructively or destructively interfere, such that the total amplitude falls below a threshold level at lower depths such that it avoids activation of undesired fibers. In embodiments, this may not be necessary: the resonant frequencies of some Aβ fibers, as well as the required amplitudes, will not overlap the resonant frequencies and amplitudes of Aδ or C fibers. In such a case, use of a single central electrode (cathodic or anodic, respectively) positioned approximately radially underneath the target nerve fiber on the underside of the finger together with the possible activation of one or more surrounding electrodes (anodic or cathodic, respectively) will suffice for activation of Aβ fibers. In embodiments, to increase penetration depth of the field, reinforcing or opposing electrodes provided diametrically opposite (on the upper half of the finger) may be activated. See FIG. 2, for example, which illustrates an exemplary ring electrode.

In embodiments, one or several ring electrodes 12r may be used per finger as indicated in FIG. 4. In embodiments, one ring 12r, positioned lower on the finger, may be used to selectively block activation of certain fibers while stimulation is provided by ring electrodes positioned higher on the finger.

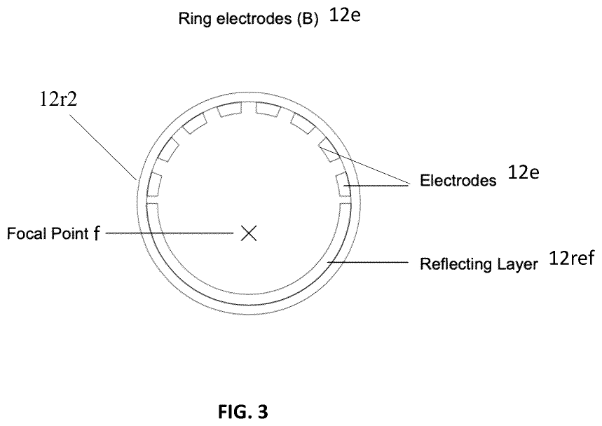

In embodiments, a second type of ring electrode 12r2 (see FIG. 3) may be used to function either on its own or in combination with the previously mentioned electrodes 12r. In embodiments, the second type of ring electrode 12r2 relies on reflection to focus electromagnetic waves to a desired depth in the user's skin. In embodiments, between one and twelve electrode elements 12e may be positioned on one half of the ring, while the other half includes a reflective metallic material 12ref provided in a circular or elliptical shape. In embodiments, by activating the electrodes elements 12e, electromagnetic waves may be focused on the center/focus f of the circle/ellipse in order to concentrate the stimulation at a desired depth in the user's skin. See FIG. 3.

In embodiments, all electrodes 12a, 12r may be incorporated into a glove. As noted above, in embodiments, the electrode array 12 may be provided on cloth such that it may be implemented as any sort of garment.

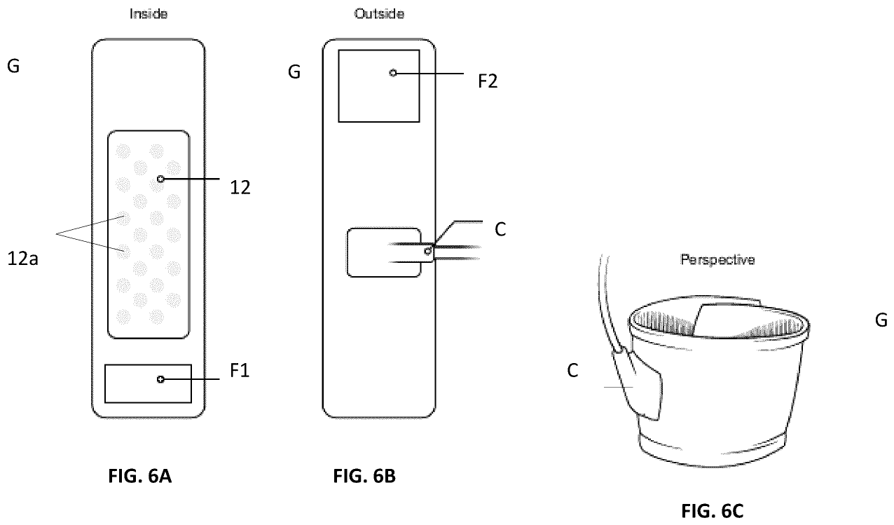

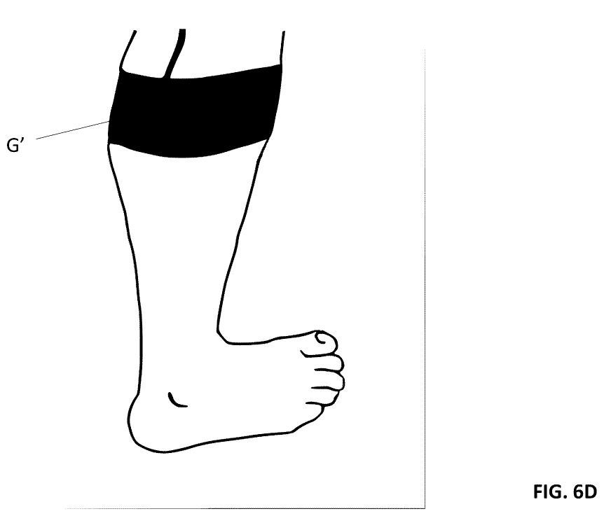

In embodiments, ring electrode configurations, as described herein, may further be used on additional body appendages, including the lower and upper limbs, and feet. FIG. 6A illustrates an inward facing side of a garment G or band on which electrode array 12 may be provided. In embodiments, the array 12 may include a plurality of electrodes 12a. In embodiments, the electrodes may be mounted in the manner discussed above with respect to FIG. 1. In embodiments, the electrodes 12a may be arranged in a different arrangement or pattern. In embodiments, the inward facing side of the garment G may include a fastening element F1. FIG. 6B illustrates an outward facing side of a garment G on which the array 12 may be mounted. In embodiments, the outward facing side includes a second fastening element F2 that may cooperate with the fastening element F1 such that the garment G may be wrapped around a leg of a patient. In embodiments, a connector C may be provided to electrically connect the array 12 to a stimulation device, such as the electrical stimulation device 14 discussed above. FIG. 6C illustrates the garment G with the fastening elements F1 and F2 connected to each other. FIG. 6D shows the garment G wrapped around a user's leg. In embodiments, the fastening elements F1, F2 may be embodied as a hook and loop type fastener. In embodiments, the fastening elements F1, F2 may utilize a snap configuration. In embodiments, the fastening elements F1, F2 may be embodied using any desired fastener.

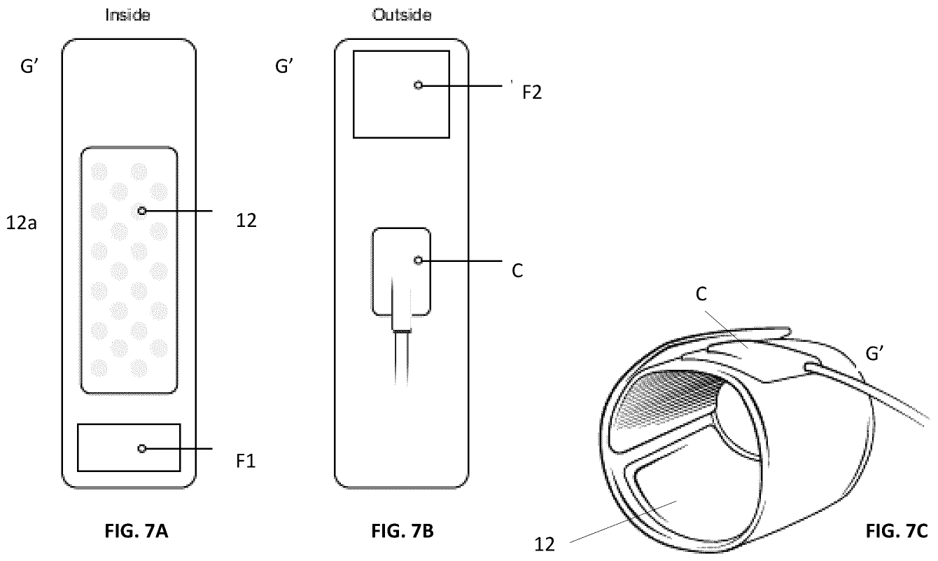

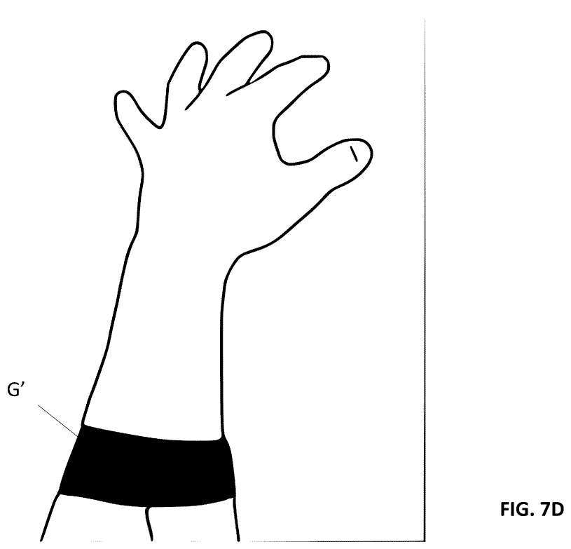

In embodiments, FIG. 7A illustrates an inward facing side of another garment G′ or band on which electrode array 12 may be provided. In embodiments, the array 12 may include a plurality of electrodes 12a. In embodiments, the electrodes may be mounted in the manner discussed above with respect to FIG. 1. In embodiments, the electrodes 12a may be arranged in a different arrangement or pattern. In embodiments, the inward facing side of the garment G′ may include a fastening element F1. FIG. 7B illustrates an outward facing side of a garment G′ on which the array 12 may be mounted. In embodiments, the outward facing side includes a second fastening element F2 that may cooperate with the fastening element F1 such that the garment G′ may be wrapped around a leg of a patient. In embodiments, a connector C may be provided to electrically connect the array 12 to a stimulation device, such as the electrical stimulation device 14 discussed above. FIG. 7C illustrates the garment G′ with the fastening elements F1 and F2 connected to each other. FIG. 7D shows the garment G′ wrapped around a user's arm.

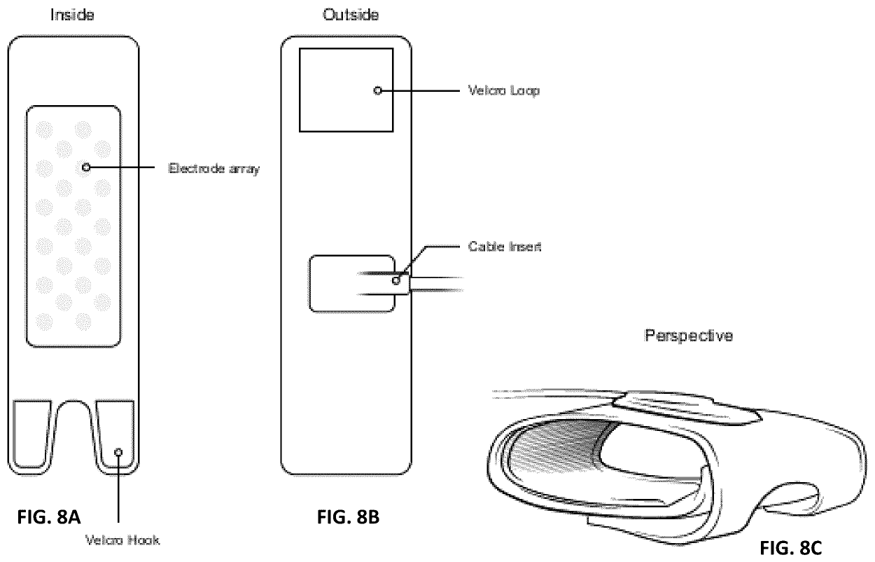

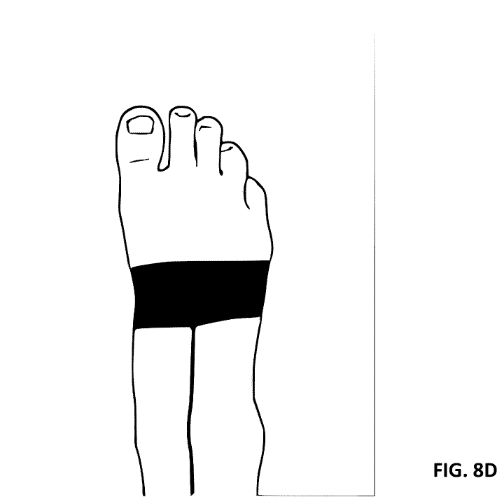

In embodiments, FIG. 8A illustrates an inward facing side of another garment G″ or band on which electrode array 12 may be provided. In embodiments, the array 12 may include a plurality of electrodes 12a. In embodiments, the electrodes may be mounted in the manner discussed above with respect to FIG. 1. In embodiments, the electrodes 12a may be arranged in a different arrangement or pattern. In embodiments, the inward facing side of the garment G″ may include a fastening element F1. FIG. 8B illustrates an outward facing side of a garment G″ on which the array 12 may be mounted. In embodiments, the outward facing side includes a second fastening element F2 that may cooperate with the fastening element F1 such that the garment G″ may be wrapped around a leg of a patient. In embodiments, a connector C may be provided to electrically connect the array 12 to a stimulation device, such as the electrical stimulation device 14 discussed above. FIG. 8C illustrates the garment G″ with the fastening elements F1 and F2 connected to each other. FIG. 8D shows the garment G′ wrapped around a user's foot.

### Stimulator

In embodiments, the electronic stimulation device 14 may be rechargeable and portable and may include dual connectors or ports to provide for simultaneous stimulus waveforms to multiple electrode arrays 12. In embodiments, the electronic stimulation device 14 may generate a wide range of waveform types such as: pulse signals, ramp signals, rectangular signals, trapezoidal signals, sinusoidal signals and half sinusoidal signals.

In embodiments, the electronic stimulation device 14 offers customizable stimulation settings, including a broad range of current intensities {0.01-30 mA} with a resolution of 10 μA or greater. In embodiments, the frequency range spans from 0 to 1 MHz.

### Stimulation Settings

### Aδ Fibers: Different Stimulation Settings may be Provided for Three Types of Aδ Fiber

In embodiments, a first type of fiber involved with touch and nociception may be associated with Piezo2 and TRPM8 channel receptors: may be stimulated with a central cathode in area B, possibly surrounded by anodes, that stimulates with a half sinusoidal, sinusoidal, pulse, constant or other variable signal frequency of between 1 and 10 Hz, at 0.01 mA to 3 mA, or with a central electrode with a sinusoidal frequency of 5 Hz, with surrounding electrodes at lower current amplitude stimulating at the same frequency, but exactly 180 degrees out of phase with the central electrode.

In embodiments, a second type of Aδ fiber is a proprioceptive Aδ fiber associated with Piezo2 channels: may be stimulated with a central cathode in Area B, possibly surrounded by anodes, stimulates with a half sinusoidal frequency of 250 Hz, at 0.1-5 mA, or with a central electrode with a sinusoidal frequency of 250 Hz, with surrounding electrodes at lower current amplitude stimulating at the same frequency, but 180 degrees out of phase with the central electrode.

Nociceptive Aδ, a third type of Aδ fiber may be associated with TRPM8 channels and sharp or fast pain perception: may be stimulated with a square anodic pulse of amplitude 0.1-5 mA at 50-100 Hz which is followed by a cathodic pulse of lower amplitude (0.1-4 mA) at a lower frequency. Alternatively, sinusoidal stimulation may be used in the same frequency range, with surrounding electrodes 180 degrees out of phase, and possibly with the negative part of the sinusoidal curve having lower amplitude than the positive part.

### C Fibers: Different Stimulation Settings may be Used for Three Types of C Fibers

Touch/nociceptive fibers, a first type of C fiber may be characterized by MRGPRA3 and HRH1 receptors: may be stimulated by a central anode in Area B stimulating at 1-10 Hz in a half-sinusoidal waveform surrounded by cathodes stimulating at a lower amplitude at the same frequency, with a half-sinusoidal waveform. Alternatively, the central electrode in embodiments, stimulates sinusoidally and the surrounding electrodes stimulate sinusoidally 180 degrees out of phase with the central electrode, at a lower amplitude where the amplitude of any electrode is between 0.01 mA and 1 mA.

Nociceptive fibers, a second type of C fiber, may be characterized by TRPV1 channel receptors: may be stimulated by a central anode in Area B stimulating at 0.5-5 Hz in a half-sinusoidal waveform, with one half period pause between positive sinusoidal half-periods, surrounded by cathodes stimulating at a lower amplitude at the same frequency, with a half-sinusoidal waveform, possibly with one half period between positive sinusoidal half-periods. Alternatively, the central electrode may stimulate half sinusoidally, possible with a half-period pause, and the surrounding electrodes stimulate sinusoidally 180 degrees out of phase with the central electrode, at a lower amplitude, and also possible with a half-period pause. In embodiments, the amplitude of any electrode is between 0.1 mA and 5 mA.

Nociceptive fibers, a third type of C fiber, may be characterized by Piezo2 channel receptors: may stimulate a central anode stimulating at 2-10 Hz in a half-sinusoidal waveform, (Area B) possibly with one half period pause between positive sinusoidal half-periods, may be surrounded by cathodes stimulating at a lower amplitude at the same frequency, with a half-sinusoidal waveform, possibly with one half-period pause between positive sinusoidal half-periods. Alternatively, the central electrode may stimulate half sinusoidally, possible with a half-period pause, and the surrounding electrodes may stimulate sinusoidally 180 degrees out of phase with the central electrode, at a lower amplitude, and also possibly with a half-period pause. In embodiments, the amplitude of any electrode is between 0.05 mA and 10 mA

Aβ fibers may be divided into two types of rapid/fast adapting fibers (RAI and RAII) and two types of slowly adapting fibers (SAI and SAII). The system 10 of the present disclosure allows for the stimulation of RAI, RAII and SAI fibers as follows.

To selectively activate RAI fibers through electrical stimulation, in embodiments, a sinusoidal/half sinusoidal, square or ramp waveforms, or a sequence of pulses, with a frequency ranging from 5 to 80 Hz and central anodic polarity is applied, possibly with surrounding anodal polarities, with the specific amplitude between one half and five times the perception threshold. This may be applied in Areas A, B or C of the pad 12 B including rectangular electrode array 12, corresponding to the location of the desired stimulation. To simulate sensations corresponding to edge contours or slippage, different distributed electrode clusters in the ring may be activated in a pulse-like fashion, with an anodal center electrode and a cathode surrounding it. In embodiments, the electrodes may be activated in a specific sequence to simulate the desired sensation. In order to prevent nociceptive fibers from activating, in embodiments, the rectangular electrode array 12 may be used to create a fiber block.

In embodiments, to selectively activate RA2 fibers through electrical stimulation, a sinusoidal/half sinusoidal or square waveform, or a sequence of pulse trains, with a frequency ranging from 10 to 250 Hz and central cathode with possible anodic surround is applied, with specific one half and five times the perception threshold, via Areas A, B or C of the rectangular electrode array. In embodiments, sensations corresponding to high-frequency vibrations or tapping may be induced by electrodes in the rings 14r activated with the same waveforms, with a cathodal center and anodal surround, with the location of the electrodes being activated varying according to the desired sensation.

In embodiments, to selectively activate SAI fibers through electrical stimulation, a sinusoidal/half sinusoidal or ramped waveform, or a sequence of pulses, with a frequency ranging from 1 to 10 Hz and central cathodic polarity with possible anodal surround is applied, with an amplitude of 0.05 to 1 mA, via Areas A, B or C of the rectangular electrode array. In embodiments, the rise-time of the applied signal (in the case of a rectangular waveform) may be varied to be between one-tenth and twice that of the constant part of the pulse duration.

In addition, in embodiments, the stimulation of fibers, the electrode arrays 12 may also be used to block certain afferent signals. In embodiments, to effectively stimulate only C fibers, it may be necessary to block signals from simultaneously activated Aδ fibers. This may be done with a cathodic ramp current with a duration of 10-50 ms, to be repeated as necessary while other fibers are being activated.

### Stimulation at Locations

In embodiments, the electrode configuration allows for stimulation of fibers that are not situated directly underneath the principal, central electrode. In embodiments, the current of each electrode may be individually controlled by the electronic stimulation device 14, hence by increasing/decreasing the current to the central electrode and decreasing/increasing the current to any or all of the surrounding electrodes in the rectangular/patch including array 12 in an individual fashion, the electric field may be shaped in such a way as to activate fibers situated between electrodes.

In embodiments, biosignals may be acquired to provide efficacy monitoring and closed-loop feedback control of various electrical stimulation waveforms provided by the electronic stimulation device 14. In embodiments, local muscle responses to electrical stimulation may be recorded via transcutaneous electromyography (EMG)/acceleromyography (AMG), while cortical responses to electrical stimulation may also be recorded via electroencephalography (EEG). In embodiments, EMG/AMG channels may include up to four local muscles/limbs, depending on the stimulation site. In embodiments EEG may include up to 24 channels, with frontal, central, parietal, occipital, and temporal electrodes utilized-along with mastoid and reference electrodes as appropriate.

In embodiments, integration of biofeedback signal may be obtained using EMG/AMG and/or EEG with transcutaneous electrical stimulation provided by the electronic stimulation device 14. In embodiments, electrical stimulation may be delivered with and without biosignal feedback.

In embodiments, selective stimulation of Aβ, Aδ, and C cutaneous sensory afferents, via aforementioned embodiments, may be verified by numerous sources of sensory biofeedback, included in further embodiments. Electromyography (EMG) objectively measures electrical activity of muscles and may be used in conjunction with targeting electrical stimuli to acquire reflex responses to both noxious and non-noxious stimuli. In embodiments, EMG electrodes associated with muscles associated with electrical stimulation regions, for example, the biceps femoris and tibialis anterior muscles of the leg during transcutaneous stimulation of the foot sole enable monitoring of tightly linked noxious and non-noxious reflex loops of the lower limb, respectively. In embodiments, acceleromyography (AMG) technology may also be used to acquire motion information associated with limbs, in response to electrical stimulation. In embodiments, acquiring overall system responses rather than individual muscle responses may be preferred, when complex reflexive patterns occur in response to electrical stimulation such as in noxious stimulation of the hands, for example. In embodiments, reflexive responses used to verify selective activation of Aβ, Aδ, and C cutaneous sensory afferents may include outcomes such as F-waves, H-reflex, CMAPs, along with RII and RIII reflexes acquired via EMG and AMG, depending on the location of transcutaneous electrical stimulation and stimulation waveform.

In embodiments, electroencephalography (EEG) may be used to verify selective activation of Aβ, Aδ, and C cutaneous sensory afferents, as well as monitor responsiveness to progressive stimulation paradigms. EEG acquires electrical activity from sensors adorned on the scalp, and is related to underlying brain activity both at rest and in response to varying stimuli. In embodiments, as few as 4 and as many as 20 EEG sensors may be used, with at least one sensor located in the central region, at least one located in the frontal region, at least one located in the parietal region, and at least one in the temporal region, depending on the specific embodiment. In embodiments, EEG embodiments include resting and evoked potential outcomes, namely the use of power spectra frequency bands at rest, and both time and time-frequency responses to evoked stimuli. Regarding resting state EEG outcomes, examination of power spectra frequency before and after various stimulation protocols, in specific embodiments, enables the indirect assessment of anxiety, attention, and other factors that contribute to pain experience. Here, in embodiments, the use of combined resting and evoked EEG outcomes, enables both the verification of selective activation of Aβ, Aδ, and C cutaneous sensory afferents, as well as monitoring of pain-related biomarkers for verification of analgesia for therapeutic end points in other embodiments.

In embodiments, integration of biofeedback using EMG, AMG and/or EEG with the electronic stimulation device 14 may be achieved through integration with a stimulation controller or processing hub. In embodiments, the stimulation controller may be provided in the electronic stimulation device 14. In embodiments, the stimulation controller may be provided separate from the electronic stimulation device 14, for example, as part of the EMG, AMG or EEG system. In embodiments, the stimulation controller may be provided outside of the electronic stimulation device 14 and the EMG, AMG or EEG system, for example, in a computer device such as a PC or a server or on a mobile computer device such as a smart phone, laptop computer or tablet computer. In embodiments, the stimulation controller may receive the stimulation waveform along with the biofeedback information to monitor a response of the user to stimulation waveforms and provide adjustment information that may be used to adjust the stimulation waveform to ensure activation of the proper fiber. Examples of such biofeedback may rely on EMG/AMG based outcomes, such as tactile and nociceptive reflexes, whereby the latency and amplitudes of responses are indicative of stimulus intensity, as precepted by peripheral sensory fibers. Further examples include sensory evoked potentials, acquired from EEG. Here, specific electrode configurations, along with specific stimulation profiles, can be used to monitor tactile and pain related responses to evoked stimuli.

In some embodiments, the number of active stimulating electrodes required per stimulation type previously described may expand and contract in accordance with principles of spatial summation, namely that less intense stimuli from multiple sensory fibers in close spatial proximity may induce similar responsiveness as more intense stimuli over a more precise, targeted area. Some embodiments will employ switching between focused and expansive waveform protocols to leverage spatial summation for various nerve locations or nerve density applications. By leveraging aforementioned biofeedback mechanisms, the ability to locate nerves/characterizing peripheral nerve density through these methods of expanding and contracting electric fields, is also within the scope of the current invention. In some embodiments, the temporal responsiveness of peripheral nerves may also be used to verify receptor subtype. Namely, nociceptive afferents have temporal summation responsiveness, whereby repetitive stimuli at specific frequencies (0.01 Hz-3 Hz) result in amplified responses in the spinal cord, and subsequent supraspinal centers. In such embodiments, both EMG/AMG and EEG based outcomes paired with repetitive stimulation will verify temporal summation phenomenon, as a further classification of peripheral nerve receptor subtype.

In FIG. 5, an exemplary electrical stimulation device 14 is illustrated as part of the system 10, which provides multiple stimulating outputs 141, 142, 143, each of which may be connected to an electrode array 12 in contact with the patient or to one or more ring electrodes as noted above. In embodiments, each of the outputs 141, 142, 143 may provide a different stimulation waveform depending on the application of each array of electrodes. In embodiments, a feedback input 144 may be provided and connected to one or more of an EMG, AMG or EEG system, which may be included in the multiple biological signal acquisition systems discussed above that gather biosignals from the user to provide feedback information regarding the user's reaction to the stimulation waveforms. In embodiments, the feedback information may be provided to a stimulation controller 14b which may be a CPU including a microcontroller or another suitable control device. In embodiments, the microcontroller 14n may receive the feedback information and may provide the stimulation waveforms to the outputs 141, 143 and 143. In embodiments, the microcontroller 14b may include or be connected to a bluetooth module, or other communication module, which may be configured to provide information to an external device, and/or to receive information from an external device. In embodiments, control information may be provided from an external device, such as a mobile electronic device, smartphone, laptop computer or tablet, to name a few, which may be used to generate the stimulation waveforms, to process the feedback information and/or to update the stimulation waveform based on processing of the feedback information. In embodiments, the electronic stimulation device 14 incudes a power source which may be used to provide power to the microcontroller 14b and to provide the stimulating waveforms. In embodiments, the power source may be, or include, a battery, preferably a rechargeable battery and associated charging circuitry. In embodiments, a D/A converter may be provided to convert control signals from the microcontroller to analog stimulation waveforms to be provided to arrays 12 to the outputs 141, 142, 143. In embodiments, different waveforms may be provided to the respective outputs depending on the fibers to be activated and their relative location in the body.

Although the present invention is described and shown in relation to particular embodiments thereof, many other variations and modifications and other uses will become apparent to those skilled in the art. Thus, various embodiments and variations are shown and described herein, and it is preferred, therefore, that the present invention be limited not by the specific disclosure herein.

What is claimed is:

1. A system for activating desired nerve fibers comprises:
an electrode array configured for contact with a user's body;
an electrical stimulation device electrically connected to the electrode array to provide electrical signals to the electrode array having desired currents and voltages;
a biological sensor configured to collect biological information associated with the user and provides it to the electrical stimulation device as feedback information;
wherein the desired currents and voltages are applied to the user's body to stimulate desired nerve fibers and the feedback information indicates user reaction to the desired currents and voltages.

2. The system of claim 1, wherein the biological sensor includes at least one of a group consisting of an Electromyography (EMG) system collecting EMG information associated with muscle activity and an electroencephalography (EEG) system collecting EEG information associated with the users peripheral nervous system.

3. The system of claim 1, wherein the electrode array includes
a first type of electrode; and
a second type or electrode,
wherein the first type of electrode is provided with a first electrical signal from the electrical stimulation device providing a first current and a first voltage configured to stimulate a first type of nerve fiber; and
the second type of electrode is provided with a second signal from the electrical stimulation device providing a second current and a second voltage configured to stimulate a second type of nerve fiber.

4. The system of claim 1, wherein the electrode array includes:
a first area including the first type of electrode;
a second area including the second type of electrode; and
a third area including the first type of electrode.

5. The system of claim 1, wherein first type of electrode is hemispherical in shape and the second type of electrode is conical in shape with a sharp tip that pierces the user's skin.

6. The system of claim 5, wherein the first type of electrode is configured to selective activate large touch fiber subtypes (Aβ), of small, myelinated fibers (Aδ) and the second type of electrode is configured to selectively activate small unmyelinated nerve fibers (C fibers).

7. The system of claim 6, wherein, in the first area and the second area, a central electrode within a first cluster of electrodes that acts as a cathode and surrounding electrodes provide anodic stimulation.

8. The system of claim 7, wherein varying current distribution is provided to the surrounding electrodes to activate nerve fibers not situated directly underneath the central electrode.

9. The system of claim 8, wherein the first area and third area include multiple clusters of electrodes to increase a number of nerve fibers being activated.

10. The system of claim 9, wherein the first type of electrode induces a pre-conditioning fiber block when a ground electrode is positioned opposite the stimulated area.

11. The system of claim 6, wherein the second type of electrodes in the second area includes a first grouping of the second type of electrodes including one electrode designated as an anode and surrounding electrodes acting as cathodes.

12. The system of claim 11, wherein the second type of electrode in the second area is used to block certain fibers.

13. The system of claim 5, wherein the first electrode type is provided with a first signal having a variable signal frequency of between 1 and 10 Hz, at 0.01 mA to 3 mA.

14. The system of claim 5, wherein the first electrode type is provided with a first signal with a half sinusoidal frequency of 250 Hz, at 0.1-5 mA.

15. The system of claim 5, wherein the first electrode type is provided with a first signal with a square anodic pulse of amplitude 0.1-5 mA at 50-100 Hz followed by a cathodic pulse of lower amplitude and lower frequency.

16. The system of claim 5, wherein the second electrode type is provided with a second signal with a half-sinusoidal waveform having a frequency between 2 and 10 Hz.

17. The system of claim 5, wherein the first electrode type is provided with a first signal having a sinusoidal, half sinusoidal, square or ramp waveform or pulse sequence with a frequency ranging from 5 to 80 Hz.

18. The system of claim 5, wherein the first electrode type is provided with a first signal having a sinusoidal, half sinusoidal, square or ramp waveform or pulse sequence with a frequency ranging from 10 to 250 Hz.

19. The system of claim 5, wherein the first electrode type is provided with a first signal having a sinusoidal, half sinusoidal, square or ramp waveform or pulse sequence with a frequency ranging from 1 to 10 Hz with an amplitude of 0.05 to 1 mA.

20. The system of claim 1, wherein the electrode array is provided in a rectangular configuration.

21. The system of claim 20, wherein the electrode array is provided on a rectangular patch configured for attachment to a body of the user.

22. The system of claim 21, wherein the rectangular patch is configured to wrap around a body part of the user.

23. The system of claim 1, wherein the electrode array is provided on a ring.

24. The system of claim 23, wherein the electrodes are provided on an inner surface of the ring and spaced equally around the inner circumference of the ring such that they contact a user when the user wears the ring.

25. The system of claim 23, wherein a first type of electrode is provided on a bottom side of the ring and a second type of electrode is provided on a top side of the ring, wherein the second type of electrode is a reflective electrode to reflect electromagnetic radiation of the first type of electrode into the user's body.

26. The system of claim 25, wherein the second type of electrode is shaped to reflect electromagnetic radiation to a desired depth in the user's skin.
